(19) United States
(12) Patent Application Publication
(10) Pub. No.: US 2014/0053842 A1
(43) Pub. Date: Feb. 27, 2014

(54) MEDICAL OXYGEN MIXING VALVE

(71) Applicants: Wei Yang, Beijing (CN); Huimin Bi, Beijing (CN)

(72) Inventors: Wei Yang, Beijing (CN); Huimin Bi, Beijing (CN)

(73) Assignee: Beijing Aeonmed Co., Ltd., Beijing (JP)

(21) Appl. No.: 14/000,867

(22) PCT Filed: Dec. 25, 2012
(86) PCT No.: PCT/CN2012/087361

§ 371 Date: Aug. 21, 2013

(30) Foreign Application Priority Data
Dec. 30, 2011 (CN) 201110458100.5

(51) Int. Cl. A61M 16/12; A61M 16/20

(52) U.S. Cl. CPC A61M 16/12; A61M 16/20
USPC 128/205.24

(57) ABSTRACT

Disclosed is a medical oxygen mixing valve comprising a valve body, a valve cap, a spring, spool assembly, dial assembly and knob assembly. An axial hole is set inside the valve body. The spool components capable of sliding in an axial direction along the wall of the hole are set in the hole. An air input hole, oxygen input hole and gas mixture output hole which are in communication with the hole are set in the radial direction of the valve body. The knob components are used for adjusting the position of the spool components in the hole set in the axial direction of the valve body to adjust the sizes of the air input hole and oxygen input hole blocked by the spool components, thereby adjusting the output of the oxygen concentration. The oxygen mixing valve has the advantages of a simple structure, and easy machining, installation and debugging.

### FIELD OF THE INVENTION

The present invention relates to a valve, and particularly to a medical oxygen mixing valve.

### BACKGROUND OF THE INVENTION

A medical oxygen mixing valve is an apparatus for mixing oxygen and air by manually adjusting a knob and then outputting the mixed air with a certain percentage of oxygen. Presently, the oxygen mixing valve generally has problems of a complicated structure, difficult installation and debugging, a demanding machining process, etc.

### SUMMARY OF THE INVENTION

An object of the present invention is to provide a medical oxygen mixing valve to solve the problems of a complicated structure, difficult machining, and installation and debugging present in the existing oxygen mixing valve.

The above object of the present invention can be realized by the following technical solutions.

A medical oxygen mixing valve includes a valve body, a valve cap, a spring, a spool assembly, a dial assembly and a knob assembly, where a through hole for receiving the spool assembly is axially arranged at the center of the valve body, and an air inlet, an oxygen inlet and a mixed gas outlet, which are in communication with the through hole, are radially arranged in the valve body;

the spool assembly includes a spool and two star type sealing rings arranged on the spool, and the two star type sealing rings are used to completely close one of the air inlet and the oxygen inlet and completely open the other one of the air inlet and the oxygen inlet;

the dial assembly includes a dial disc;

the knob assembly includes a knob and an adjusting screw one end of which protrudes from the knob, and the adjusting screw is arranged in a hole axially positioned at the center of the knob;

one end of the valve body is connected with the valve cap, the other end of the valve body is connected with the dial disc, the spool assembly is arranged within the valve body, a spring for pressing against the spool is arranged between one end of the spool and the valve cap, an end of the knob where the adjusting screw protrudes from is connected with the dial disc through a projecting threaded part at the end of the knob, and the adjusting screw presses against the other end of the spool.

Preferably, the dial assembly further includes a set screw for position limiting arranged within a radial threaded hole in the dial disc, the knob assembly further includes two limiting pins arranged on the side of the knob from which the adjusting screw protrudes, an angle between two tangent lines passing through the central axis of the knob 7, one of which is tangent to a side of one of the two stop pins that is opposite to the other one of the two stop pins and the other one of which is tangent to a side of the other one of the two stop pins that is opposite to the one of the two stop pins, is 90° or 270°, and the limiting pins extend into an annular groove axially arranged on an upper part of the dial disk, and are blocked and limited by the set screw due to the rotation of the knob.

Preferably, the spool assembly further includes an O-shaped sealing ring, one end of the spool that is pressed against the adjusting screw has a hemispherical shape, and the O-shaped sealing ring is arranged on the spool at a position close to the hemispherical end.

Preferably, the valve cap and the valve body are connected through a screw and are further sealedly connected by a step and an O-shaped sealing ring.

Preferably, each of the air inlet, the oxygen inlet and the mixed gas outlet is provided with a joint.

Preferably, the valve body is connected with the dial disc through a screw.

Preferably, the knob assembly further includes a set screw, which is arranged in a radial threaded hole of the knob and is tightly pressed against the adjusting screw.

Preferably, the valve body is made of polymethyl methacrylate.

The operating principle of the present invention is described as below. An axial hole is arranged within the valve body, the spool assembly which is axially slidable along the wall of the axial hole is arranged in the axial hole, and the air inlet, the oxygen inlet and the mixed gas outlet, which are in communication with the axial hole, are radially arranged in the valve body, so that the adjustment on the oxygen concentration in the mixed gas outputted from the mixed gas outlet can be realized by the degree of blocking the air inlet and the oxygen inlet by the spool assembly when it slides within the valve body.

The present invention is advantageous for its simple structure, easy machining and convenient installation and debugging.

### BRIEF DESCRIPTION OF THE DRAWINGS

The present invention will be further described in detail below with reference to the accompanying drawings and embodiments.

FIG. 1 is a schematic front half sectional view showing the structure of a medical oxygen mixing valve according to an embodiment of the present invention.

FIG. 2 is a schematic sectional view showing the structure of the medical oxygen mixing valve taken along a line A-A shown in FIG. 1.

FIG. 3 is a schematic front sectional view showing the structure of a dial assembly used for the medical oxygen mixing valve shown in FIG. 1.

FIG. 4 is a schematic sectional view showing the structure of the dial assembly taken along a line B-B shown in FIG. 3.

FIG. 5 is a schematic diagram showing the perspective structure of a knob assembly used for the medical oxygen mixing valve shown in FIG. 1.

FIG. 6 is a schematic top view showing the structure of the knob assembly shown in FIG. 5.

FIG. 7 is a schematic sectional view showing the structure of the knob assembly taken along a line C-C shown in FIG. 5.

A list of reference numerals:

| 1: | Valve body; | 2: Valve cap; | 3: Spring; | 4: Spool; |
|---|---|---|---|---|
| 5: | Star-type | 6: Dial disc; | 7: Knob; | 8: Adjusting screw; |
| | sealing ring; | | | |
| 9: | Set screw; | 10: Limiting pin; | 11: O-shaped sealing ring; | |
| 12: | Joint; | 13: Set screw. | | |

### DETAILED DESCRIPTION OF THE EMBODIMENTS

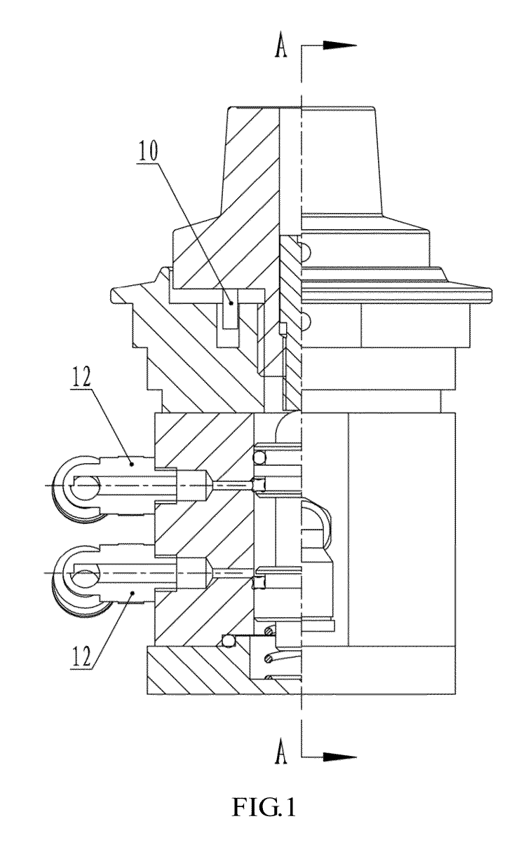

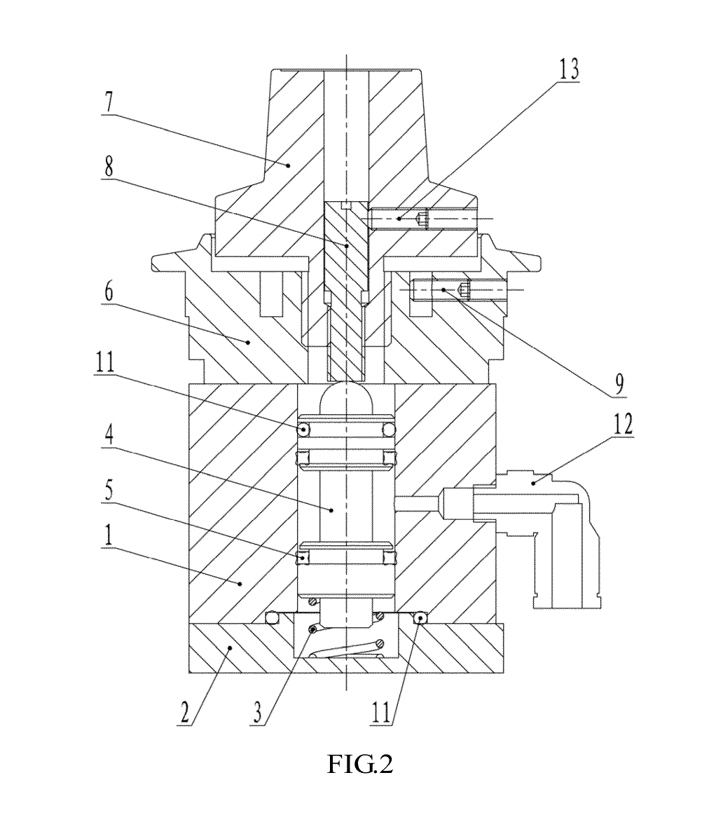

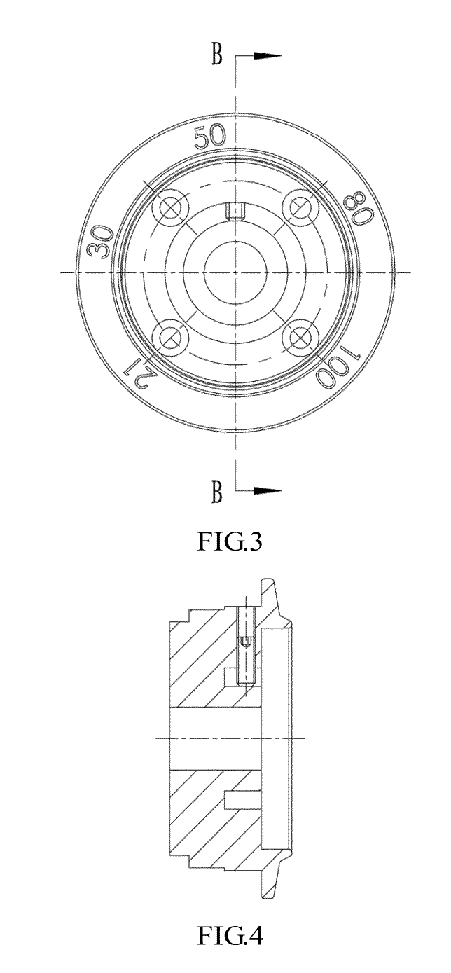

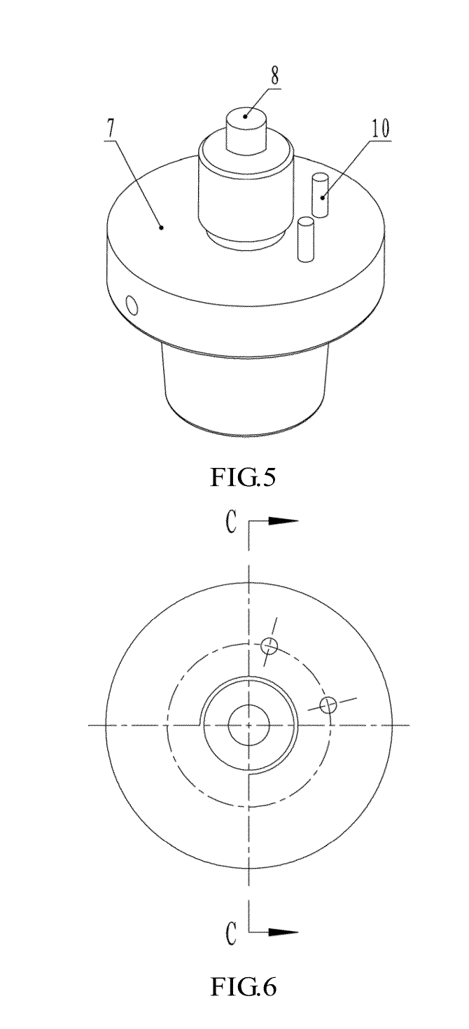

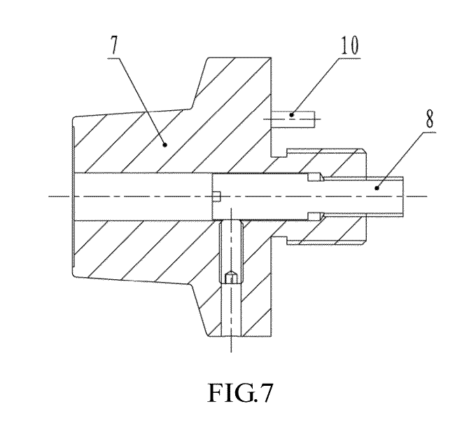

As shown in FIGS. 1 to 7, a medical oxygen mixing valve of an embodiment includes a valve body 1, a valve cap 2, a spring 3, a spool assembly, a dial assembly and a knob assembly. A through hole for receiving the spool assembly is axially arranged at the center of the valve body 1, and an air inlet, an oxygen inlet and a mixed gas outlet, which are in communication with the through hole, are radially arranged in the valve body 1.

The spool assembly includes a spool 4 and two star-type sealing rings 5 arranged on the spool, and the two star-type sealing rings 5 can be used for completely closing one of the air inlet and the oxygen inlet while completely opening the other one of the air inlet and the oxygen inlet.

The dial assembly includes a dial disc 6.

The knob assembly includes a knob 7 and an adjusting screw 8. The adjusting screw 8, one end of which protrudes from the knob 7, is arranged in a hole axially provided at the center of the knob 7.

One end of the valve body 1 is connected with the valve cap 2, and the other end of the valve body 1 is connected with the dial disc 6. The spool assembly is arranged within the valve body 1, and a spring 3 for pressing against the spool 4 is arranged between one end of the spool 4 and the valve cap 2. An end of the knob 7, where the adjusting screw 8 protrudes from, is connected with the dial disc 6 through a projecting threaded part at the end, and the adjusting screw 8 presses against the other end of the spool 4.

The dial assembly further includes a set screw 9 for position limiting, which is arranged within a radial threaded hole in the dial disc 6. Moreover, the knob assembly further includes two limiting pins 10 arranged on the side of the knob 7 from which the adjusting screw 8 protrudes, and an angle between two tangent lines passing through the central axis of the knob 7, one of which is tangent to a side of one of the two stop pins that is opposite to the other one of the two stop pins and the other one of which is tangent to a side of the other one of the two stop pins that is opposite to the one of the two stop pins, is 90° or 270°. The limiting pins 10 extend into an annular groove axially arranged on an upper part of the dial disc 6, and may be blocked and limited by the set screw 9 due to the rotation of the knob 7.

The spool assembly further includes an O-shaped sealing ring 11, one end of the spool 4 that is pressed against the adjusting screw 8 has a hemispherical shape, and the O-shaped sealing ring 11 is arranged on the spool 4 at a position close to the hemispherical end.

The valve cap 2 and the valve body 1 are connected through a screw, and are further sealedly connected by a step and another O-shaped sealing ring 11.

Each of the air inlet, the oxygen inlet and the mixed gas outlet is provided with a joint 12.

The valve body 1 is connected with the dial disc 6 through a screw.

The knob assembly further includes a set screw 13, which is arranged within a radial threaded hole in the knob 7 and is tightly pressed against the adjusting screw 8.

The valve body 1 may be made of polymethyl methacrylate.

In the present embodiment, the adjustment to the relative positions between the star type sealing ring 5 on the spool 4 and the oxygen inlet as well as the air inlet in the valve body 1 can be achieved by merely the adjusting screw 8 arranged in the knob 7, and the set screw 9 is used to block and limit the two limiting pins 10, so that the knob 7 can be rotated up to 270°. For example, when an indicator on the knob 7 is directed at a value 21 on the dial disc 6, the spring is in a compressed state, the oxygen inlet is completely closed by the star sealing ring 5, and the air inlet is opened, thus the oxygen concentration of the mixed gas outputted by the mixed gas outlet is 21%. Then, if the knob 7 is rotated sequentially in a clockwise direction, the oxygen concentration is increased gradually. When the spool assembly is moved to such a position that the air inlet is completely closed by the other star sealing ring 5, the oxygen inlet is opened, and the oxygen concentration of the mixed gas output by the mixed gas outlet is 100%.

The present invention solves the problems of a complicated structure, difficult machining as well as difficult installation and debugging present in the existing oxygen mixing valve, and is advantageous for its simple structure, easy machining as well as convenient installation and debugging.

What is claimed is:

1. A medical oxygen mixing valve, comprising a valve body, a valve cap, a spring, a spool assembly, a dial assembly and a knob assembly, wherein a through hole for receiving the spool assembly is axially arranged at the center of the valve body, and an air inlet, an oxygen inlet and a mixed gas outlet, which are in communication with the through hole, are radially arranged in the valve body;
the spool assembly comprises a spool and two sealing rings arranged on the spool, and the two sealing rings are used to completely close one of the air inlet and the oxygen inlet and completely open the other one of the air inlet and the oxygen inlet;
the dial assembly comprises a dial dis;
the knob assembly comprises a knob and an adjusting screw one end of which protrudes from the knob, and the adjusting screw is arranged in a hole axially positioned at the center of the knob;
one end of the valve body is connected with the valve cap, the other end of the valve body is connected with the dial disc, the spool assembly is arranged within the valve body, a spring for pressing against the spool is arranged between one end of the spool and the valve cap, an end of the knob where the adjusting screw protrudes from is connected with the dial disc through a projecting threaded part at the end of the knob, and the adjusting screw presses against the other end of the spool.

2. The medical oxygen mixing valve of claim 1, wherein, the two sealing rings are star-typed.

3. The medical oxygen mixing valve of claim 1, wherein, the dial assembly further comprises a set screw for position limiting arranged within a radial threaded hole in the dial disc, the knob assembly further comprises two limiting pins arranged on the side of the knob from which the adjusting screw protrudes, and the limiting pins extend into an annular groove axially arranged on an upper part of the dial disk, and are blocked and limited by the set screw due to the rotation of the knob.

4. The medical oxygen mixing valve of claim 2, wherein, the spool assembly further comprises an O-shaped sealing ring, one end of the spool that is pressed against the adjusting screw has a hemispherical shape, and the O-shaped sealing ring is arranged on the spool at a position close to the hemispherical end.

5. The medical oxygen mixing valve of claim 1, wherein, the valve cap and the valve body are connected through a screw and are further sealedly connected by a step and an O-shaped sealing ring.

6. The medical oxygen mixing valve of claim 1, wherein, each of the air inlet, the oxygen inlet and the mixed gas outlet is provided with a joint.

7. The medical oxygen mixing valve of claim 1, wherein, the valve body is connected with the dial disc through a screw.

8. The medical oxygen mixing valve of claim 1, wherein, the knob assembly further comprises a set screw, which is arranged in a radial threaded hole of the knob and is tightly pressed against the adjusting screw.

9. The medical oxygen mixing valve of claim 1, wherein, the valve body is made of polymethyl methacrylate.

10. The medical oxygen mixing valve of claim 1, wherein, a rotational angle of the knob is in a range from 0° to 270.
